(19) United States
(12) Patent Application Publication
(10) Pub. No.: US 2006/0134202 A1
(43) Pub. Date: Jun. 22, 2006

(54) Homeopathic sublingual dosage forms and methods thereof

(76) Inventors: Jacob C. Hack, Las Vegas, NV (US); Richard D. Tobias, Las Vegas, NV (US)

Correspondence Address: WEISS & MOY PC, 4204 NORTH BROWN AVENUE, SCOTTSDALE, AZ, 85251, US

(21) Appl. No.: 11/022,093

(22) Filed: Dec. 22, 2004

(51) Int. Cl. A61K 9/20

(52) U.S. Cl. 424/464

(57) ABSTRACT

A sublingual dosage form comprises an inert preformed tablet including at least one excipient and at least one wicking agent, with the at least one wicking agent adapted to selectively absorb at least one solvent-borne medicating ingredient from one portion of the inert preformed tablet into another portion of the inert preformed tablet without disintegrating the inert preformed tablet. The inert preformed tablet is medicated with at least one homeopathic ingredient. A person ingests the sublingual dosage form which disintegrates in about 25 seconds to about 120 seconds after placement under a tongue or a buccal area of a mouth of the person. The sublingual dosage form has a friability of less than about 2% and a hardness of about 3 Newtons to about 7 Newtons.

### FIELD OF THE INVENTION

This invention relates generally to tablets, and, more specifically, to homeopathic sublingual dosage forms and methods thereof in tablet form.

### BACKGROUND OF THE INVENTION

Homeopathic sprays, tablet triturates and compressed tablets are preferably ingested by insertion into the buccal pouch or sublingually where tablet triturates and sublingual tablets rapidly dissolved and absorbed. Currently, homeopathic tablets must be marked with a manufacturer's identification, which may be a coded indentation in a portion of the compressed tablet. Tablet triturates cannot be coded with a mark because of the size and manufacturing method for tablet triturates.

A homeopathic medicated tablet having no protective outer coating may be produced by impregnating (medicating) an inert tablet with at least one drug attenuation (very low dosage homeopathic ingredient dissolved in a solvent), such as ethanol. The inert portion of the tablet comprises at least one excipient such as lactose, sucrose or a mixture of lactose and sucrose, at least one of a binder and a lubricant and the like, to provide a formulation with the homeopathic ingredient dissolved in the solvent, which may be compressed into a tablet form. The sublingual tablet so formed is dried at a temperature not exceeding 40° C. to drive off a substantial portion of the solvent.

During impregnation of inert sublingual tablet ingredients, it is important to maintain the physical structure, appearance and marked indentation coding of the sublingual tablet. A sublingual tablet must also readily disintegrate when placed under a person's tongue or buccal area of the person's mouth. The hardness of such a sublingual tablet is a compromise between being too hard and too soft. If the sublingual tablet is too hard, the solvent borne drug attenuation may not be absorbed into an interior portion of the tablet and therefore remains on a surface portion of the tablet, where the drug attenuation may not adhere to the sublingual tablet. If the sublingual tablet is too soft, the sublingual tablet may be disintegrated by the solvent of the drug attenuation. Preferably, the solvent borne drug attenuation should be absorbed into the interior of the sublingual tablet. However, it has proven difficult to impregnate an inert sublingual tablet by addition of solvent borne drug attenuations.

Kankari et al., U.S. Pat. No. 6,024,981 disclosed a rapidly dissolving robust dosage form (sublingual tablet) comprising an active ingredient mixed into a matrix of a non-direct compression filler and a relatively high lubricant content. The Kankari et al. sublingual tablet may comprise a wicking agent, which acts as a disintegrating agent when the tablet is orally consumed, and the tablet is made by blending the active ingredient with the matrix components and compressing a tablet form, which differs from the current invention. Similarly, Capella, U.S. Pat. No. 6,500,456 disclosed a compressed nitroglycerine sublingual tablet made by blending a nitroglycerine/lactose dilution with hydrous lactose, glyceril monostearate, fumed silica, pregelatinized starch and calcium stearate and compressing this blend to form a sublingual tablet, which differs from the current invention. Neither tablet form as disclosed by either Kankari et al. or Capella (above) are made by impregnating a inert preformed tablet with an active ingredient.

The current inventors have determined that incorporation of a suitable absorbent (alternatively termed a wicking agent, because solvent is believed to be drawn in by capillary action) into an inert sublingual tablet permits effective impregnation of a solvent borne attenuation into an interior portion of the inert preformed sublingual tablet while maintaining the structural integrity of the medicated sublingual tablet.

### SUMMARY OF THE INVENTION

Accordingly, it is an object of this disclosure to provide a sublingual dosage form comprising an inert preformed tablet adapted to selectively absorb at least one solvent-borne medicating ingredient without disintegrating the inert preformed tablet.

It is a further object of this disclosure to provide a sublingual dosage form comprising an inert preformed tablet adapted to selectively absorb at least one solvent-borne homeopathic ingredient without disintegrating the inert preformed tablet.

It is a still further object of this disclosure to provide a substantially non-friable sublingual dosage form comprising an inert preformed tablet adapted to selectively absorb at least one solvent-borne homeopathic ingredient without disintegrating the inert preformed tablet and which rapidly disintegrates after placement under a tongue or a buccal area of a mouth of a person.

It is a further object of this disclosure to provide a method for manufacturing a sublingual dosage form comprising an inert preformed tablet adapted to selectively absorb at least one solvent-borne medicating ingredient without disintegrating the inert preformed tablet.

It is a still further object of this disclosure to provide a method for manufacturing a sublingual dosage form comprising an inert preformed tablet adapted to selectively absorb at least one solvent-borne homeopathic ingredient without disintegrating the inert preformed tablet.

It is a yet further object of this disclosure to provide a method for dosing a person with a sublingual dosage form comprising an inert preformed tablet adapted to selectively absorb at least one solvent-borne homeopathic ingredient without disintegrating the inert preformed tablet.

### PREFERRED EMBODIMENTS OF THE INVENTION

In accordance with one embodiment of this invention, a sublingual dosage form is disclosed. The sublingual dosage form comprises, in combination, an inert preformed tablet including at least one excipient and at least one wicking agent, the at least one wicking agent adapted to selectively absorb at least one solvent-borne medicating ingredient from one portion of the inert preformed tablet into another portion of the inert preformed tablet without disintegrating the inert preformed tablet. The medicating ingredient comprises at least one homeopathic ingredient, at least a portion of the solvent being evaporated from the inert preformed tablet after absorption of the homeopathic ingredient into the another portion of the inert preformed tablet thereby both retaining the shape and medicating the inert preformed tablet to provide the sublingual dosage form.

In accordance with a second embodiment of this invention, a method for manufacturing a sublingual dosage form is disclosed. The method comprises the steps of providing at least one solvent-borne medicating ingredient in a solvent; providing an inert preformed tablet including at least one excipient and at least one wicking agent, the at least one wicking agent adapted to selectively absorb the at least one solvent-borne medicating ingredient from one portion of the inert preformed tablet into another portion of the inert preformed tablet; impregnating the inert preformed tablet with the at least one solvent-borne medicating ingredient; and evaporating at least a portion of the solvent from the impregnated inert preformed tablet without disintegrating the inert preformed tablet thereby both retaining the shape and medicating the inert preformed tablet to provide the sublingual dosage form.

In accordance with a third embodiment of this invention, a method for dosing a person with a sublingual dosage form is disclosed. The method comprises the steps of providing an inert preformed tablet including at least one excipient and at least one wicking agent, the at least one wicking agent adapted to selectively absorb at least one solvent-borne medicating ingredient from one portion of the inert preformed tablet into another portion of the inert preformed tablet without disintegrating the inert preformed tablet; providing the sublingual dosage form comprising the inert preformed tablet and at least one medicating ingredient absorbed by the inert preformed tablet; and ingesting the sublingual dosage form by the person, the sublingual dosage form disintegrating in about 25 seconds to about 120 seconds after placement under a tongue or a buccal area of a mouth of the person, the sublingual dosage form having a friability of less than about 2% and a hardness of about 3 Newtons to about 7 Newtons.

The foregoing and other articles, features, and advantages of the invention will be apparent from the following more detailed description of the preferred embodiments of the invention.

### DESCRIPTION OF THE INVENTION

As described above, homeopathic tablets must be marked with a manufacturer's identification, which may be a coded indentation in a portion of the compressed tablet. A homeopathic compressed tablet may be ingested sublingually by a person by inserting the tablet into the mouth or a buccal area of the mouth of the person and rapidly disintegrating and absorbing the tablet in the mouth. This procedure is particularly preferred when the homeopathic ingredient would be destroyed in the stomach. However, the former requirement mentioned above (the marking of the tablet) and the latter requirement (rapid disintegration and absorption) appear to make it difficult to produce a tablet by impregnating a pre-marked inert preformed tablet with a solvent borne medication, such as a homeopathic ingredient, without somewhat disintegrating or losing some of the structural integrity of the inert preformed tablet.

According to one embodiment of this disclosure, a sublingual dosage form comprises, in combination, an inert preformed tablet including at least one excipient and at least one wicking agent, the at least one wicking agent adapted to selectively absorb at least one solvent-borne medicating ingredient from one portion of the inert preformed tablet into another portion of the inert preformed tablet without disintegrating the inert preformed tablet. The medicating ingredient comprises at least one homeopathic ingredient, at least a portion of the solvent being evaporated from the inert preformed tablet after absorption of the homeopathic ingredient into another portion of the inert preformed tablet thereby both retaining the shape and medicating the inert preformed tablet to provide the sublingual dosage form. The solvent comprises at least one of water, ethyl alcohol and isopropyl alcohol. It is understood that mixtures of these solvents may be suitable for dissolving the at least one homeopathic ingredient and for medicating (impregnating) the inert preformed tablet. Preferably, the solvent is evaporated at a temperature of less than about 40° C. after medicating the inert preformed tablet. It is understood that the amount of the at least one homeopathic ingredient dissolved in the solvent varies according to well-known principles of Homeopathy, and as mentioned above, the inert preformed tablet is pre-marked to indicate the amount of the at least one homeopathic ingredient.

A homeopathic ingredient according to this disclosure is defined as shown in the Homeopathic Pharmacopoeia of the United States Revision Service (HPRS) December 2003. It is understood that the HPRS is regularly updated, and the medicated sublingual dosage form of this disclosure may comprise a newly developing and as yet unidentified at least one homeopathic ingredient. The term tablet as used herein includes tablets of any shape and caplets, which are tablets having a capsule shape. The term dosage form refers to a inert preformed tablet which has been medicated with at least one homeopathic ingredient. It is understood that it is possible for the dosage form to comprise any suitable medicinally active ingredient. It is further understood that while various ingredients suitable for use in this disclosure may have the suffix USP (United States Pharmacopia) or NF (National Formulary), this provides for better identification of the ingredient, or its purity, and should not be construed as a limitation of this disclosure, since identical ingredients are available under other designations in other countries.

According to this disclosure, an excipient is generally at least one of a binder, filler, lubricant and flow-controlling agent. The inert preformed tablet of the sublingual dosage form of this disclosure comprises at least one of anhydrous and hydrated forms of a sugar and a sugar alcohol selected from the group consisting of lactose, dextrose, sucrose, fructose, maltose, xylose, maltodextrin, dextrin, cyclodextrin, mannitol and sorbitol, and the like. The at least one of the sugar and the sugar alcohol comprise about 80% to about 98% by weight of the inert preformed tablet. The at least one of the sugar and the sugar alcohol provide both a pleasant taste and provide for rapid disintegration of the sublingual dosage form in the mouth of the person, while being a binder for substantially maintaining the shape of the inert preformed tablet prior to ingestion by a person.

The inert preformed tablet of the sublingual dosage form may further comprise at least one form of anhydrous silica and hydrated silica selected from the group consisting of precipitated, fumed, amorphous, colloidal and crystalline, and the like. Preferably, the anhydrous silica and the hydrated silica comprise about 0.1% to about 1.8% by weight of the inert preformed tablet. Silica is believed to provide both some degree of flow control, and also increases the hardness of the inert preformed tablet.

The inert preformed tablet of the sublingual dosage form may further comprise at least one hydrophobic lubricant including an alkali metal salt of a fatty acid selected from the group consisting of calcium stearate and magnesium stearate, and the like. Preferably, the hydrophobic lubricant comprises about 0.1% to about 1% by weight of the inert preformed tablet, and most preferably the hydrophobic lubricant comprises about 0.1% to about 0.7% by weight of the inert preformed tablet. According to the current disclosure, the hydrophobic lubricant comprising about 0.4% to about 0.7% by weight of the inert preformed tablet has been found to be advantageous.

In addition to the above-mentioned ingredients, the inert preformed tablet may comprise at least one of a wicking agent and a disintegrating agent. The wicking agent is capable of drawing up a solvent such as water, ethyl alcohol, isopropyl alcohol, mixtures thereof and the like and thereby transports the solvent into an interior portion of the inert preformed tablet. Additionally, a wicking agent may also help transport solvating components from a person's saliva into an interior of the sublingual dosage form. A wicking agent may also be a disintegrating agent, which facilitates rapid disintegration of the sublingual dosage form when a person places the sublingual dosage form into the mouth or a buccal area of the mouth of the person. A disintegrating agent may not rapidly absorb water by capillary action. It is preferable to use either at least one of a rapidly water soluble wicking agent or a disintegrating agent and to minimize the use of at least one of a generally non-water soluble wicking agent and a disintegrating agent. Preferably, the at least one wicking agent is selected from the group consisting of a polysaccharide and a polymer and the at least one wicking agent comprises about 4% to about 25% by weight of the inert preformed tablet. More preferably, the at least one wicking agent comprises about 5% to about 18% by weight of the inert preformed tablet. The at least one wicking agent, which also may be a disintegrating agent, comprises a polysaccharide selected from the group consisting of microcrystalline cellulose, croscarmellose sodium, a hydroxyalkyl cellulose including hydroxymethyl cellulose, hydroxypropyl cellulose and hydroxypropylmethyl cellulose, arabic, soy polysaccharide, xanthan, starch and sodium starch glycolate. The at least one wicking agent may also comprise a polymer selected from the group consisting of cross-linked polyvinylpyrrolidone and carbopol, and more preferably the at least one wicking agent is a cross-linked polyvinylpyrrolidone such as Crospovidone and the like. As disclosed herein, the at least one wicking agent or disintegrating agent which is rapidly water soluble can be used in greater quantity and does not add to the grittiness of the sublingual dosage form when ingested in the mouth of the person.

The sublingual dosage form has a friability of less than about 2% and a hardness of about 3 Newtons to about 7 Newtons. When compared to other homeopathic sublingual dosage forms, the sublingual dosage form of this disclosure provides optimal disintegration and significantly less friability, and thus improved structural integrity against external mechanical forces, such as those occurring during packing, shipping and handling of the sublingual dosage form. The hardness of the sublingual dosage form as a tablet substantially enables handling without risk of tablet breakage.

The sublingual dosage form of the current disclosure disintegrates in about 25 seconds to about 120 seconds after placement under a tongue or a buccal area of a mouth of a person. Most preferably the sublingual dosage form disintegrates in about 20 seconds to about 45 seconds after placement under a tongue or a buccal area of a mouth of a person.

### Method of Manufacturing a Sublingual Dosage Form

A method for manufacturing a sublingual dosage form comprises the steps of providing at least one solvent-borne medicating ingredient in a solvent followed by providing an inert preformed tablet including at least one excipient and at least one wicking agent, the at least one wicking agent adapted to selectively absorb the at least one solvent-borne medicating ingredient from one portion of the inert preformed tablet into another portion of the inert preformed tablet. The method further comprises the steps of impregnating the inert preformed tablet with the at least one solvent-borne medicating ingredient and evaporating at least a portion of the solvent from the impregnated inert preformed tablet without disintegrating the inert preformed tablet thereby both retaining the shape and medicating the inert preformed tablet to provide the sublingual dosage form.

In one embodiment of the method, the method of manufacturing the inert tablet comprises the steps of screening at least one of anhydrous and hydrated forms of a sugar and a sugar alcohol through a sieve mesh, followed by screening at least one wicking agent through a sieve mesh, adding a solvent to both of the at least one of the sugar and the sugar alcohol and the wicking agent and wet granulating both of the at least one of the sugar and the sugar alcohol and the wicking agent, drying both of the at least one of the sugar and the sugar alcohol and the wicking agent at no more than about 40° C. until both of the at least one of the sugar and the sugar alcohol and the wicking agent are substantially solvent free. Both of the at least one of the sugar and the sugar alcohol and the wicking agent are further granulated through a sieve mesh to provide a granulated mixture and silica is added to the granulated mixture and the silica and the granulated mixture is screened through a sieve. The method of manufacturing the inert tablet further comprises the steps of adding at least one alkali metal stearate to the silica and the granulated mixture and screening the at least one alkali metal stearate, the silica and the granulated mixture through a sieve mesh, and blending and compressing the at least one alkali metal stearate, the silica and the granulated mixture thereby providing the inert preformed tablet having a predetermined shape.

In a specific example of manufacturing the inert preformed tablet at least one of lactose, dextrose and sucrose comprising about 81.9% by weight of the inert preformed tablet is screened through a #18 mesh sieve and Crospovidone (a wicking agent or a disintegrating agent) is also screened through a #18 mesh sieve. Both the at least one of lactose, dextrose and sucrose and the Crospovidone comprising about 16.0% by weight of the inert preformed tablet are wet granulated as a mixture with at least one of ethyl alcohol and isopropyl alcohol, and then the mixture is placed in a drying oven at a temperature of about 39° C. until the mixture is substantially dry. The dried mixture is then dry granulated through a #18 sieve. Colloidal silica comprising about 1.4% by weight of the inert preformed tablet is screened through a #18 sieve and blended with the dried mixture for about 20 minutes. Magnesium stearate comprising about 0.7% by weight of the inert preformed tablet is also sieved through a #18 sieve and blended together with the at least one of lactose, dextrose and sucrose, the Crospovidone, and the colloidal silica for about 5 minutes thereby providing a powdered form of the inert tablet. The powdered form of the inert tablet is compressed into an inert preformed tablet having at least one marking showing the degree of strength of the liquid drug attenuation to be used to medicate the inert preformed tablet and identifying the manufacturer. The homeopathic ingredient is dissolved in a solvent of at least one of water, ethyl alcohol and isopropyl alcohol at the desired liquid drug attenuation, and then the solution of the homeopathic ingredient and the solvent is sprayed on the surface of the inert preformed tablet and the resulting medicated inert preformed tablet is dried at a temperature of no more than about 40° C. thereby providing a required sublingual dosage form.

In an alternative embodiment of the method for manufacturing the inert tablet in which no solvent is used to manufacture the inert tablet, the method comprises the steps of screening at least one of a sugar and a sugar alcohol through a sieve mesh and screening at least one wicking agent through a sieve mesh; commingling the wicking agent with the at least one of the sugar and the sugar alcohol and granulating the wicking agent and the at least one of the sugar and the sugar alcohol through a sieve mesh to provide a granulated mixture. The method for manufacturing the inert tablet further comprises the steps of adding silica to the granulated mixture and screening the silica and the granulated mixture through a sieve, adding at least one alkali metal stearate to the silica and the granulated mixture and screening the at least one alkali metal stearate, the silica and the granulated mixture through a sieve mesh, followed by blending and dry compressing the at least one alkali metal stearate, the silica and the granulated mixture thereby providing the inert preformed tablet having a predetermined shape.

The inert preformed tablet may be medicated by a number of methods. Typically, the homeopathic ingredient is dissolved in at least one of water, ethyl alcohol and isopropyl alcohol and mixtures thereof, and the like at a predetermined attenuation to form a homeopathic solution. The homeopathic solution is preferably applied on a portion of the surface of the inert preformed tablet by spraying. Alternatively, the inert preformed tablet may be placed in a container and the homeopathic solution may be added with a predetermined contact time between the homeopathic solution and the inert preformed tablet.

### Method of for Dosing a Person with a Sublingual Dosage Form

A method for dosing a person with a sublingual dosage form, comprises the steps of providing an inert preformed tablet including at least one excipient and at least one wicking agent, the at least one wicking agent adapted to selectively absorb at least one solvent-borne medicating ingredient from one portion of the inert preformed tablet into another portion of the inert preformed tablet without disintegrating the inert preformed tablet, providing the sublingual dosage form comprising the inert preformed tablet and at least one medicating ingredient absorbed by the inert preformed tablet; and ingesting the sublingual dosage form by the person, the sublingual dosage form disintegrating in about 25 seconds to about 120 seconds after placement under a tongue or a buccal area of a mouth of the person, the sublingual dosage form having a friability of less than about 2% and a hardness of about 3 Newtons to about 7 Newtons. The method further comprises the steps of providing the medicating ingredient comprises at least one homeopathic ingredient and providing the solvent comprises at least one of water, ethyl alcohol and isopropyl alcohol.

Furthermore, in conformance with the disclosure above, the method comprises the steps of providing the sublingual dosage form comprises at least one of anhydrous and hydrated forms of a sugar and a sugar alcohol selected from the group consisting of lactose, dextrose, sucrose, fructose, maltose, xylose, maltodextrin, dextrin, cyclodextrin, mannitol and sorbitol, the at least one of the sugar and the sugar alcohol comprising about 80% to about 98% by weight of the inert preformed tablet, providing the sublingual dosage form comprises at least one form of anhydrous silica and hydrated silica selected from the group consisting of precipitated, fumed, amorphous, colloidal and crystalline with the anhydrous silica and the hydrated silica comprising about 0.1% to about 1.8% by weight of the inert preformed tablet; and providing the sublingual dosage form comprises at least one of calcium stearate and magnesium stearate comprising about 0.1% to about 0.7% by weight of the inert preformed tablet. The method further comprises the steps of providing the sublingual dosage form comprising the wicking agent is selected from the group consisting of a polysaccharide and a polymer, the wicking agent comprising about 4% to about 25% by weight of the inert preformed tablet; and providing the polysaccharide is selected from the group consisting of microcrystalline cellulose, croscarmellose sodium, a hydroxyalkyl cellulose including hydroxymethyl cellulose, hydroxypropyl cellulose and hydroxypropylmethyl cellulose, arabic, soy polysaccharide, xanthan, starch and sodium starch glycolate, and the polymer is selected from the group consisting of cross-linked polyvinylpyrrolidone and carbopol.

While the invention has been particularly shown and described with reference to preferred embodiments thereof, it will be understood by those skilled in the art that the foregoing and other changes in form and details may be made therein without departing from the spirit and scope of the invention.

What is claimed is:

1. A sublingual dosage form comprising, in combination: an inert preformed tablet including at least one excipient and at least one wicking agent, said at least one wicking agent adapted to selectively absorb at least one solvent-borne medicating ingredient from one portion of said inert preformed tablet into another portion of said inert preformed tablet without disintegrating said inert preformed tablet.

2. The sublingual dosage form according to claim 1 wherein said medicating ingredient comprises at least one homeopathic ingredient, at least a portion of said solvent being evaporated from said inert preformed tablet after absorption of said homeopathic ingredient into said another portion of said inert preformed tablet thereby both retaining the shape and medicating said inert preformed tablet to provide said sublingual dosage form.

3. The sublingual dosage form according to claim 1 wherein said solvent comprises at least one of water, ethyl alcohol and isopropyl alcohol.

4. The sublingual dosage form according to claim 1 further comprising at least one of anhydrous and hydrated forms of a sugar and a sugar alcohol selected from the group consisting of lactose, dextrose, sucrose, fructose, maltose, xylose, maltodextrin, dextrin, cyclodextrin, mannitol and sorbitol, said at least one of said sugar and said sugar alcohol comprising about 80% to about 98% by weight of said inert preformed tablet.

5. The sublingual dosage form according to claim 1 further comprising at least one form of anhydrous silica and hydrated silica selected from the group consisting of precipitated, fumed, amorphous, colloidal and crystalline with said anhydrous silica and said hydrated silica comprising about 0.1% to about 1.8% by weight of said inert preformed tablet.

6. The sublingual dosage form according to claim 1 further comprising at least one hydrophobic lubricant including an alkali metal salt of a fatty acid selected from the group consisting of calcium stearate and magnesium stearate, said hydrophobic lubricant comprising about 0.1% to about 1% by weight of said inert preformed tablet.

7. The sublingual dosage form according to claim 1, wherein at least one of calcium stearate and magnesium stearate comprising about 0.1% to about 0.7% by weight of said inert preformed tablet.

8. The sublingual dosage form according to claim 1, wherein at least one of calcium stearate and magnesium stearate comprising about 0.4% to about 0.7% by weight of said inert preformed tablet.

9. The sublingual dosage form according to claim 1, wherein said wicking agent is selected from the group consisting of a polysaccharide and a polymer, said wicking agent comprising about 4% to about 25% by weight of said inert preformed tablet.

10. The sublingual dosage form according to claim 1, wherein said at least one wicking agent comprising about 5% to about 18% by weight of said inert preformed tablet.

11. The sublingual dosage form according to claim 9 wherein said polysaccharide is selected from the group consisting of microcrystalline cellulose, croscarmellose sodium, a hydroxyalkyl cellulose including hydroxymethyl cellulose, hydroxypropyl cellulose and hydroxypropylmethyl cellulose, arabic, soy polysaccharide, xanthan, starch and sodium starch glycolate, and said polymer is selected from the group consisting of cross-linked polyvinylpyrrolidone and carbopol.

12. The sublingual dosage form according to claim 1 wherein said sublingual dosage form having a friability of less than about 2%, a hardness of about 3 Newtons to about 7 Newtons and said sublingual dosage form disintegrates in about 25 seconds to about 120 seconds after placement under a tongue or a buccal area of a mouth of a person.

13. The sublingual dosage form according to claim 1 wherein said sublingual dosage form disintegrates in about 20 seconds to about 45 seconds after placement under a tongue or a buccal area of a mouth of a person.

14. A method for manufacturing a sublingual dosage form comprising the steps of:
providing at least one solvent-borne medicating ingredient in a solvent;
providing an inert preformed tablet including at least one excipient and at least one wicking agent, said at least one wicking agent adapted to selectively absorb said at least one solvent-borne medicating ingredient from one portion of said inert preformed tablet into another portion of said inert preformed tablet;
impregnating said inert preformed tablet with said at least one solvent-borne medicating ingredient; and
evaporating at least a portion of said solvent from said impregnated inert preformed tablet without disintegrating said inert preformed tablet thereby both retaining the shape and medicating said inert preformed tablet to provide said sublingual dosage form.

15. The method according to claim 14 further comprising the steps of:
screening at least one of anhydrous and hydrated forms of a sugar and a sugar alcohol through a sieve mesh;
screening at least one wicking agent through a sieve mesh;
adding a solvent to both of said at least one of sugar and sugar alcohol and said wicking agent and wet granulating both of said at least one of sugar and sugar alcohol and said wicking agent;
drying both of said at least one of said sugar and said sugar alcohol and said wicking agent at no more than about 40° C. until both of said at least one of said sugar and said sugar alcohol and said wicking agent are substantially solvent free;
granulating both of said at least one of said sugar and said sugar alcohol and said wicking agent through a sieve mesh to provide a granulated mixture;
adding silica to said granulated mixture and screening said silica and said granulated mixture through a sieve;
adding at least one alkali metal stearate to said silica and said granulated mixture and screening said at least one alkali metal stearate, said silica and said granulated mixture through a sieve mesh; and
blending and compressing said at least one alkali metal stearate, said silica and said granulated mixture thereby providing said inert preformed tablet having a predetermined shape.

16. The method according to claim 14 further comprising the steps of:
screening at least one of a sugar and a sugar alcohol through a sieve mesh and screening at least one wicking agent through a sieve mesh;
commingling said wicking agent with at least one of said sugar and said sugar alcohol;
granulating said wicking agent and at least one of said sugar and said sugar alcohol through a sieve mesh to provide a granulated mixture;
adding silica to said granulated mixture and screening said silica and said granulated mixture through a sieve;
adding at least one alkali metal stearate to said silica and said granulated mixture and screening said at least one alkali metal stearate, said silica and said granulated mixture through a sieve mesh; and
blending and dry compressing said at least one alkali metal stearate, said silica and said granulated mixture thereby providing said inert preformed tablet having a predetermined shape.

17. A method for dosing a person with a sublingual dosage form, comprising the steps of:
providing an inert preformed tablet including at least one excipient and at least one wicking agent, said at least one wicking agent adapted to selectively absorb at least one solvent-borne medicating ingredient from one portion of said inert preformed tablet into another portion of said inert preformed tablet without disintegrating said inert preformed tablet;
providing said sublingual dosage form comprising said inert preformed tablet and at least one medicating ingredient absorbed by said inert preformed tablet; and
ingesting said sublingual dosage form by said person, said sublingual dosage form disintegrating in about 25 seconds to about 120 seconds after placement under a tongue or a buccal area of a mouth of said person, said sublingual dosage form having a friability of less than about 2% and a hardness of about 3 Newtons to about 7 Newtons.

18. The method according to claim 17 further comprising the steps of:
providing said medicating ingredient comprises at least one homeopathic ingredient; and
providing said solvent comprises at least one of water, ethyl alcohol and isopropyl alcohol.

19. The method according to claim 17 further comprising the steps of:
providing said sublingual dosage form comprises at least one of anhydrous and hydrated forms of a sugar and a sugar alcohol selected from the group consisting of lactose, dextrose, sucrose, fructose, maltose, xylose, maltodextrin, dextrin, cyclodextrin, mannitol and sorbitol, said at least one of said sugar and said sugar alcohol comprising about 80% to about 98% by weight of said inert preformed tablet;
providing said sublingual dosage form comprises at least one form of anhydrous silica and hydrated silica selected from the group consisting of precipitated, fumed, amorphous, colloidal and crystalline with said anhydrous silica and said hydrated silica comprising about 0.1% to about 1.8% by weight of said inert preformed tablet; and
providing said sublingual dosage form comprises at least one of calcium stearate and magnesium stearate comprising about 0.1% to about 0.7% by weight of said inert preformed tablet.

20. The method according to claim 17 further comprising the steps of:
providing said sublingual dosage form comprising said wicking agent is selected from the group consisting of a polysaccharide and a polymer, said wicking agent comprising about 4% to about 25% by weight of said inert preformed tablet; and
providing said polysaccharide is selected from the group consisting of microcrystalline cellulose, croscarmellose sodium, a hydroxyalkyl cellulose including hydroxymethyl cellulose, hydroxypropyl cellulose and hydroxypropylmethyl cellulose, arabic, soy polysaccharide, xanthan, starch and sodium starch glycolate, and said polymer is selected from the group consisting of cross-linked polyvinylpyrrolidone and carbopol.
